(19) United States
(12) Patent Application Publication
(10) Pub. No.: US 2012/0180168 A1
(43) Pub. Date: Jul. 12, 2012

(54) METHOD FOR PRODUCING DOUBLE HAPLOID PLANTS

(76) Inventor: Robert Hélène Ghislain Dirks, Oudenbosch (NL)

(21) Appl. No.: 13/350,163

(22) Filed: Jan. 13, 2012

Related U.S. Application Data
(63) Continuation-in-part of application No. PCT/EP2010/060076, filed on Jul. 13, 2010.

(30) Foreign Application Priority Data
Jul. 14, 2009 (EP) 09165440.0

(51) Int. Cl. A01H 5/00; C12N 5/04; A01H 5/10

(52) U.S. Cl. 800/299; 435/410

(57) ABSTRACT

The present invention relates to a method for producing double haploid plants, which may comprise the steps of allowing pollen with one functional sperm cell to fertilize an embryo sac cell which is not the central cell; allowing the central cell to proliferate into endosperm; and regenerating a double haploid plant from the endosperm. The pollen with one functional sperm cell may be for example mutant pollen, which is obtainable by chemical mutation, transformation with a nucleic acid, or irradiation.

### RELATED APPLICATIONS AND INCORPORATION BY REFERENCE

This application is a continuation-in-part application of international patent application Ser. No. PCT/EP2010/060076 filed 13 Jul. 2010, which published as PCT Publication No. WO 2011/006899 on 20 Jan. 2011, which claims benefit of European patent application Ser. No. 09165440.0 filed 14 Jul. 2009.

The foregoing applications, and all documents cited therein or during their prosecution (“appln cited documents”) and all documents cited or referenced in the appln cited documents, and all documents cited or referenced herein (“herein cited documents”), and all documents cited or referenced in herein cited documents, together with any manufacturer's instructions, descriptions, product specifications, and product sheets for any products mentioned herein or in any document incorporated by reference herein, are hereby incorporated herein by reference, and may be employed in the practice of the invention. More specifically, all referenced documents are incorporated by reference to the same extent as if each individual document was specifically and individually indicated to be incorporated by reference.

### FIELD OF THE INVENTION

The present invention relates to a new method of producing double haploid plants. The invention further relates to plants thus obtained, and to progeny, cells, tissues and seeds of these plants.

### BACKGROUND OF THE INVENTION

Since the discovery by Guha & Maheshwari in 1964 (Nature 204: 497) that plants can be regenerated from haploid spores, a lot of research has been done to obtain similar knowledge for other species (see e.g. “In vitro Haploid production in Higher plants” Vol. 1, 2, 3, 4, 5, Eds: S. Jain, S. Sopory and R. Veilleux (1996) Kluwer Academic Publishers).

In modern, contemporary plant breeding the use of double haploids (DHs) has become a very valuable tool in order to speed up the creation of genetically pure lines and also to evaluate and monitor difficult traits such as those that are encoded by multiple genes/alleles.

The production and the use of DHs in breeding crop plants is well known for many species (see e.g. Thomas W. et al. (2003), In: Doubled haploid production in crop plants. A Manual. Eds. M. Maluszynski, K. Kasha, B. Forster and I. Szarejko. Kluwer Academic Publishers, pp 337-349). Thus far, DHs can be obtained from spores of the male or female organs. Spores from the male organs are called microspores and the in vitro cultures are called microspore cultures. Typical microspore cultures are well established in Brassica since a long time (see e.g. Keller et al. (1984) In: K. Giles, S. Sen (eds.), Plant Cell Culture in Crop Improvement pp 169-183. Plenum Pub. Corp., New York). Spores from the female organs are called megaspores, and the in vitro culture of these spores is commonly named gynogenesis. Gynogenesis is a well established technique for e.g. sugar beet and also cucumber (see e.g. Hosemans D. and Bossoutrot, Z. Pflanzenzuchtg. 91:74-77 (1983); EP 0 374 755).

The success of both gynogenesis and microspore cultures is despite many technological advancements only limited to amenable genotypes. Not only are there plant species with low success rate for creating DHs such as watermelon (Sari N., Hort. Science 1994, vol. 29(10), 1189-1190) and squash (Kurtar E. S. et al., Euphytica, Volume 127(3), 2002, 335-344 (10), some species are completely recalcitrant for induction of DHs.

This means that the enormous benefits of DHs cannot be exploited in every desired plant species.

Citation or identification of any document in this application is not an admission that such document is available as prior art to the present invention.

### SUMMARY OF THE INVENTION

It is therefore the object of the present invention to provide a new method for producing DHs. This object is achieved by a method for producing a double haploid (DH) plant which may comprise:
a) allowing pollen with one functional sperm cell to fertilize an embryo sac cell which is not the central cell;
b) allowing the central cell to proliferate into endosperm;
c) and regenerating a double haploid plant from the endosperm.

The present invention does not obtain DH plants directly from using micro- or megaspores. Instead, the DH plant is regenerated from the central cell of the female gametophyte. Accordingly, the invention involves, a method for producing a double haploid plant, comprising regenerating the double haploid plant from endosperm proliferated from the central cell after pollen with one functional sperm cell fertilizes an embryo sac cell which is not the central cell.

While not prior art to the instant invention, mention is made of Chen et al., The Central Cell Plays a Critical Role in Pollen Tube Guidance in Arabidopsis, The Plant Cell 19:3563-3577 (2007) and Liu et al., Development and function of central cell in angiosperm female gametophyte, Genesis. 2010 Aug;48(8):466-78.

Accordingly, it is an object of the invention to not encompass within the invention any previously known product, process of making the product, or method of using the product such that Applicants reserve the right and hereby disclose a disclaimer of any previously known product, process, or method. It is further noted that the invention does not intend to encompass within the scope of the invention any product, process, or making of the product or method of using the product, which does not meet the written description and enablement requirements of the USPTO (35 U.S.C. §112, first paragraph) or the EPO (Article 83 of the EPC), such that Applicants reserve the right and hereby disclose a disclaimer of any previously described product, process of making the product, or method of using the product.

It is noted that in this disclosure and particularly in the claims and/or paragraphs, terms such as “comprises”, “comprised”, “comprising” and the like can have the meaning attributed to it in U.S. Patent law; e.g., they can mean “includes”, “included”, “including”, and the like; and that terms such as “consisting essentially of” and “consists essentially of” have the meaning ascribed to them in U.S. Patent law, e.g., they allow for elements not explicitly recited, but exclude elements that are found in the prior art or that affect a basic or novel characteristic of the invention.

These and other embodiments are disclosed or are obvious from and encompassed by, the following Detailed Description.

### BRIEF DESCRIPTION OF THE DRAWINGS

The following detailed description, given by way of example, but not intended to limit the invention solely to the specific embodiments described, may best be understood in conjunction with the accompanying drawings.

FIG. 1 illustrates a method of the present invention. An embryo sac cell 1 contains three antipodal cells 2, a dinuclei central cell 3 and a haploid egg cell 4 flanked by two synergids 5 and 6. When fertilization 7 takes place with wild type pollen 8 with two functional sperm cells 9 and 10 a fertilized triploid central egg cell 11 and a fertilized diploid egg cell 12 are formed in the embryo sac cell 1. Upon germination a diploid plant 13 is formed from the embryo. After fertilization 16 with mutant pollen 14, which contains only one functional sperm cell 15, no fertilization of the central cell 17 takes place. The unfertilized central cell 17 is double haploid. The egg cell 18 is diploid after fertilization. A double haploid plant 19 can subsequently be regenerated from the central cell 17.

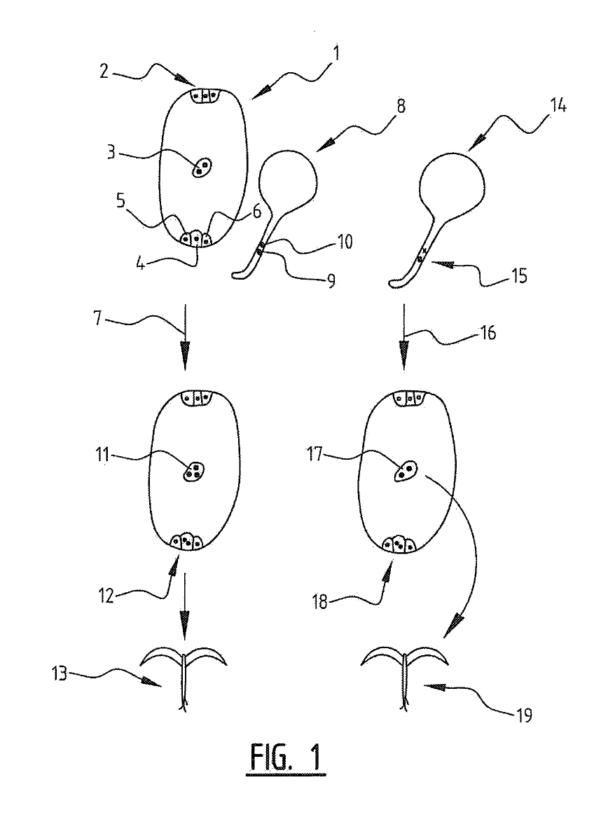

### DETAILED DESCRIPTION OF THE INVENTION

Sexual reproduction in Angiosperms is characterized by a unique process called double fertilization. This means that the two sperm cells from the pollen grain enter the female gametophyte. The first sperm cell will then fertilize the haploid egg cell and the second will fertilize the central cell which contains two nuclei. From the fertilized egg cell a diploid embryo will develop, and from the central cell triploid endosperm will proliferate. Without fertilization of the central cell, and/or without a trigger from the fertilized egg cell the central cell will generally not proliferate into endosperm. The only exceptions are fis and fie mutants that can give autonomous endosperm development.

In the present invention, however, mutant pollen in which one of the sperm cells is absent or inactivated will only fertilize the egg cell. The central cell will be left unfertilized in the absence of a second sperm cell, and thus remains in the diploid stage, which is in essence a double haploid. Fertilization of the egg cell will trigger the proliferation of the unfertilized central cell into endosperm. From there on techniques for regenerating triploid plants out of endosperm, widely available for many plant species, can be used (see T. D. Thomas & R. Chaturvedi, Plant Cell Tissue and Organ Culture 93: 1) to regenerate double haploid plants from the unfertilized central cell.

The invention thus relates to the use of mutant pollen for the fertilization of the egg cell only, which may trigger the development of the unfertilized double haploid central cell.

In one embodiment pollen with only one functional sperm cell may be created by chemical mutagenesis with EMS (ethane methyl sulfonate) or chemicals such as, but not limited to, sulphonates such as EES (ethyl ethane sulphonate), BMS (butyl methanesulphonate), PMS (propyl methanesulphonate), MES (methyl ethane sulphonate), or MMS (methyl methanesulphonate).

In one embodiment pollen with only one functional sperm cell may be created by mutagenesis via irradiation using e.g. UV light, X-ray, gamma ray, or ionizing radiation.

In one embodiment mutagen plants may be screened for the appropriate mutation, being inhibition of cell division in the generative cell, using eco-tilling (see, e.g., Henikoff et al 2004, Plant Physiology Preview May 21, 2004).

In one embodiment natural populations may be screened for having pollen with only one functional sperm cell, using eco-tilling (see, e.g., Henikoff et al 2004, Plant Physiology Preview May 21, 2004).

In one embodiment, molecules inhibiting the division of the generative cell may be transiently expressed during the development of the pollen, for example by a nucleic acid which is present on a plasmid. The inhibiting molecules, which may be either nucleic acid or protein, may be produced in the pollen or microspores by constitutive expression from the plasmid.

In one embodiment, the molecules inhibiting the division of the generative cell may be expressed from a nucleic acid that is stably incorporated in the pollen genome. The cell division inhibiting molecules, which may be either nucleic acid or protein, may be produced in the pollen or microspores by constitutive expression.

According to one embodiment of the invention, pollen containing only one functional sperm cell may be obtainable by transformation with a nucleic acid. The transformation may be performed in any suitable way, such as by means of Agrobacterium tumefaciens or by means of particle bombardment (biolistics).

These transformation techniques are well known. Transformation of plant cells by means of Agrobacterium tumefaciens is well established and for example reviewed in De la Riva et al., EJB Vol. 1(3) (1998), and Bent, Plant Physiol. 124:1540-1547 (2000). An advantage of the technique is that DNA can be introduced into whole plant tissues, thereby bypassing the need for regeneration of an intact plant from a protoplast. Agrobacterium transformation vectors are capable of replication in E. coli as well as Agrobacterium, allowing for convenient manipulations. Moreover, advances in vectors for Agrobacterium-mediated gene transfer have improved the arrangement of genes and restriction sites in the vectors to facilitate the construction of vectors capable of expressing various polypeptide coding genes. The vectors have convenient multi-linker regions flanked by a promoter and a polyadenylation site for direct expression of inserted polypeptide coding genes. Additionally, Agrobacterium containing both armed and disarmed Ti genes can be used for transformation. In those plant strains where Agrobacterium-mediated transformation is efficient, it is the method of choice because of the facile and defined nature of the gene locus transfer. The use of Agrobacterium-mediated plant integrating vectors to introduce DNA into plant cells, including plant plant cells, is well known in the art (See, e.g., U.S. Pat. Nos. 7,250,560 and 5,563,055).

Recently, it was discovered that genetic transformation of plants is not solely restricted to Agrobacterium, but that other bacteria too have the capacity to transform plants (Broothaerts et al., Nature 433, 629-633 (2005), incorporated herein by reference). These plant-associated symbiotic bacteria were made competent for gene transfer by acquisition of both a disarmed Ti plasmid and a suitable binary vector. Such transformation systems are also suitable for use in the invention.

A particularly efficient method for delivering transforming DNA segments to plant cells is microprojectile bombardment. In this method, particles are coated with nucleic acids and delivered into cells by a propelling force. Exemplary particles include those comprised of tungsten, platinum, and preferably, gold. For the bombardment, cells in suspension are concentrated on filters or solid culture medium. Alternatively, immature embryos or other target cells may be arranged on solid culture medium. The cells to be bombarded are positioned at an appropriate distance below the macroprojectile stopping plate. An illustrative embodiment of a method for delivering DNA into plant cells by acceleration is the Biolistics Particle Delivery System, which can be used to propel particles coated with DNA or cells through a screen, such as a stainless steel or Nytex screen, onto a surface covered with target plant cells. The screen disperses the particles so that they are not delivered to the recipient cells in large aggregates. It is believed that a screen intervening between the projectile apparatus and the cells to be bombarded reduces the size of projectiles aggregate and may contribute to a higher frequency of transformation by reducing the damage inflicted on the recipient cells by projectiles that are too large. Microprojectile bombardment techniques are widely applicable, and may be used to transform virtually any plant species.

Biolistic transformation is also well known to the person skilled in the art and tools for such applications are commercial available since several years (Ralph Bock, In: QiagenNews, Issue No. 5, 1997). Suitable techniques for use in the invention are for example also described by Barinova et al. (J Exp Bot. 53(371):1119-29 (2002)), in which delivery of DNA at the level of microspores and transient expression thereof in Antirrhinum majus is shown, or by Ramaiah et al. (Current Science 73:674-682 (1997)) for alfalfa (Medicago sativa L.). Methodology for microspore or pollen transformation with biolistic bombardment in tobacco can be found in Baubak Bajoghli (Matrikel number: 9802743, University of Vienna, Experimentelle Genetic III. Plant Biotechnology by Alisher Touraev, July 2001). Van der Leede-Plegt, et al., Transgenic Research 4(2):77-86 (1995) describe direct delivery of DNA into pollen of tobacco (Nicotiana glutinosa) by means of microprojectile bombardment. These and other techniques can be used for the transformation of pollen or microspores for use in the invention.

Vectors used for the transformation of plant cells are not limited so long as the vector can express an inserted DNA in the cells. For example, vectors comprising promoters for constitutive gene expression in plant cells (e.g., cauliflower mosaic virus 35S promoter) and promoters inducible by exogenous stimuli can be used. Examples of suitable vectors include pBI binary vector. The “plant cell” into which the vector is to be introduced includes various forms of plant cells, such as cultured cell suspensions, protoplasts, leaf sections, and callus. A vector can be introduced into plant cells by known methods, such as the polyethylene glycol method, polycation method, electroporation, Agrobacterium-mediated transfer, particle bombardment and direct DNA uptake by protoplasts. To effect transformation by electroporation, one may employ either friable tissues, such as a suspension culture of cells or embryogenic callus or alternatively one may transform immature embryos or other organized tissue directly. In this technique, one would partially degrade the cell walls of the chosen cells by exposing them to pectin-degrading enzymes (pectolyases) or mechanically wound tissues in a controlled manner.

In one embodiment, the pollen and microspores thus may comprise the cell division inhibiting molecules by virtue of the presence of a nucleic acid. The nucleic acid that is introduced may be the cell division inhibiting molecule itself, or may encode the cell division inhibiting molecule. In the latter case the inhibiting molecule may be a protein or a peptide. In the first case the inhibiting molecule may be a nucleic acid. The nucleic acid may be inhibiting in itself or it can block other nucleic acids from being expressed. For example, the nucleic acid may be or code for a RNAi against members of the CDK (cyclin dependent kinase) protein family or the KRP (CDK inhibitor protein) family.

In the eukaryotic cell cycle a key role is played by the cyclin-dependent kinases (CDKs). A “cyclin dependent kinase” or “CDK” are art recognized terms referring to protein of the family of proteins which include catalytic subunits of cyclin/CDK complexes. Exemplary CDK proteins include CDC2, CDK2, CDK3, CDK4, CDK5, CDK6 and CDK7. CDK complexes are formed via the association of a regulatory cyclin subunit and a catalytic kinase subunit. In mammalian cells, the combination of the kinase subunits (such as cdc2, CDK2, CDK4 or CDK6) with a variety of cyclin subunits (such as cyclin A, B1, B2, D1, D2, D3 or E) results in the assembly of functionally distinct kinase complexes. The coordinated activation of these complexes drives the cells through the cell cycle and ensures the fidelity of the process (Draetta, Trends Biochem. Sci. 15:378-382, 1990; Sherr, Cell 73:1059-1065, 1993). Each step in the cell cycle is regulated by a distinct and specific cyclin-dependent kinase. For example, complexes of CDK4 and D-type cyclins govern the early G1 phase of the cell cycle, while the activity of the CDK2/cyclin E complex is rate limiting for the G1 to S-phase transition. The CDK2/cyclin A kinase is required for the progression through S-phase and the cdc2/cyclin B complex controls the entry into M-phase (Sherr, Cell 73:1059-1065, 1993).

The term “KRP protein” or “CKI protein” refers to a protein which can bind to and inhibit activation of a cyclin dependent kinase. Exemplary KRP or CKI proteins include members of the INK4 family and members of the CIP family. The term “INK4 protein” refers to a family of structurally related CDK inhibitors characterized by a fourfold repeated ankyrin-like sequence (Elledge et al. (1994) Curr. Opin. Cell Biol. 6:874-878), and the ability to bind to CDKs, especially CDK4 and CDK6. Exemplary members of this protein family include p16 (INK4A/MTS1; Serrano et al (1993) Nature 366:704-707); p15 (INK4B; Hamnon et al. (1994) Nature 371:257-261); p18 (Guan et al. (1994) Genes Der. 8:2939-2952) and p19 (Chan et al. (1995) Mol. Cell Biol. 15:2682-2688; and Hirai et al. (1995) Mol. Cell Biol. 15:2672-2681). Other proteins have been identified in the art as having tandemly arranged ankyrin-like sequences, such as the Pho81p protein (Ogawa et al. (1995) Mol. Cell Biol. 15:997-1004), and may provide CDK-binding motifs which are functionally equivalent to those of an INK4 protein. The term “CIP protein” refers to members of another CKI protein family which includes p21.sup.CIP1 (WAF1/SDI1/CAP20; Xiong et al. (1983) Nature 36:701-704); p27.sup.KIP1 (Polyak et al. (1994) cell 78:67-74); and p57.sup.KIP2 (Lee et al. (1995) Genes Dev. 9:639-649; and Matsuoka et al. (1995) Genes Dev. 9:650-662). In addition to the functional characteristic of CDK inhibition, the CIP proteins each have a CDK inhibitory motif (a CDK-binding motif) of about 50 amino acids, referred to herein as a “p21/p27” inhibitory domain, which is conserved in members of the CIP family, as well as, for example, members of the Rb-like protein family.

The invention is based on the principle that only one sperm cell is delivered to the embryo sac or egg cell by means of transformed or natural mutant pollen. Gene constructs or molecules that are capable of inhibiting cell division in the generative cell are in itself known and can be used in the new method of the invention.

Transformation of plant protoplasts also can be achieved using methods based on calcium phosphate precipitation, polyethylene glycol treatment, electroporation, and combinations of these treatments.

A number of promoters have utility for plant gene expression for any gene of interest including but not limited to selectable markers, scoreable markers, genes for pest tolerance, disease resistance, nutritional enhancements and any other gene of agronomic interest. Examples of constitutive promoters useful for plant gene expression include, but are not limited to, the cauliflower mosaic virus (CaMV) P-35S promoter, a tandemly duplicated version of the CaMV 35S promoter, the enhanced 35S promoter (P-e35S), the nopaline synthase promoter, the octopine synthase promoter, the figwort mosaic virus (P-FMV) promoter (see U.S. Pat. No. 5,378,619), an enhanced version of the FMV promoter (P-eFMV) where the promoter sequence of P-FMV is duplicated in tandem, the cauliflower mosaic virus 19S promoter, a sugarcane bacilliform virus promoter, a commelina yellow mottle virus promoter, the promoter for the thylakoid membrane proteins from lettuce (psaD, psaF, psaE, PC, FNR, atpC, atpD, cab, rbcS) (see U.S. Pat. No. 7,161,061), the CAB-1 promoter from lettuce (see U.S. Pat No. 7,663,027), the promoter from maize prolamin seed storage protein (see U.S. Pat No. 7,119,255), and other plant DNA virus promoters known to express in plant cells. A variety of plant gene promoters that are regulated in response to environmental, hormonal, chemical, and/or developmental signals can be used for expression of an operably linked gene in plant cells, including promoters regulated by (1) heat, (2) light (e.g., pea rbcS-3A promoter, maize rbcS promoter, or chlorophyll a/b-binding protein promoter), (3) hormones, such as abscisic acid, (4) wounding (e.g., wunl, or (5) chemicals such as methyl jasmonate, salicylic acid, or Safener. It may also be advantageous to employ organ-specific promoters.

Exemplary nucleic acids which may be introduced to the plant of this invention include, for example, DNA sequences or genes from another species, or even genes or sequences which originate with or are present in plant species, but are incorporated into recipient cells by genetic engineering methods rather than classical reproduction or breeding techniques. However, the term “exogenous” is also intended to refer to genes that are not normally present in the cell being transformed, or perhaps simply not present in the form, structure, etc., as found in the transforming DNA segment or gene, or genes which are normally present and that one desires to express in a manner that differs from the natural expression pattern, e.g., to over-express. Thus, the term “exogenous” gene or DNA is intended to refer to any gene or DNA segment that is introduced into a recipient cell, regardless of whether a similar gene may already be present in such a cell. The type of DNA included in the exogenous DNA can include DNA which is already present in the plant cell, DNA from another plant, DNA from a different organism, or a DNA generated externally, such as a DNA sequence containing an antisense message of a gene, or a DNA sequence encoding a synthetic or modified version of a gene.

Many hundreds if not thousands of different genes are known and could potentially be introduced into the plant of the present invention. Non-limiting examples of particular genes and corresponding phenotypes one may choose to introduce into a plant include one or more genes for insect tolerance, pest tolerance such as genes for fungal disease control, herbicide tolerance, and genes for quality improvements such as yield, nutritional enhancements, environmental or stress tolerances, or any desirable changes in plant physiology, growth, development, morphology or plant product(s).

Alternatively, the DNA coding sequences can affect these phenotypes by encoding a non-translatable RNA molecule that causes the targeted inhibition of expression of an endogenous gene, for example via antisense- or cosuppression-mediated mechanisms. The RNA could also be a catalytic RNA molecule (i.e., a ribozyme) engineered to cleave a desired endogenous mRNA product. Thus, any gene which produces a protein or mRNA which expresses a phenotype or morphology change of interest is useful for the practice of the present invention. (See also U.S. Pat No. 7,576,262, “Modified gene-silencing RNA and uses thereof.”)

The invention further relates to propagation material for producing plants of the invention. Such propagation material comprises inter alia seeds of the claimed plant and parts of the plant that are involved in sexual reproduction. Such parts are for example selected from the group consisting of seeds, microspores, pollen, ovaries, ovules, embryo sacs and egg cells. In addition, the invention relates to propagation material comprising parts of the plant that are suitable for vegetative reproduction, for example cuttings, roots, stems, cells, protoplasts.

According to a further aspect thereof the propagation material of the invention comprises a tissue culture of the claimed plant. The tissue culture comprises regenerable cells. Such tissue culture can be derived from leaves, pollen, embryos, cotyledon, hypocotyls, meristematic cells, roots, root tips, anthers, flowers, seeds and stems. (See generally U.S. Pat No. 7,041,876 on lettuce being recognized as a plant that can be regenerated from cultured cells or tissue).

In one embodiment pollen grains may be subsequently transferred onto the pistils of plants from the same species or a species in which pollen discharge of the said pollen/microspore cells may occur. The latter is called heterologous pollination. An example of heterologous pollination is the use of a species belonging to the Solanaceae family as a pollen donor and tomato as an acceptor. Other examples are described in de Martinis, D et al. Planta 214(5):806-812 (2002) and Dore C et al., Plant Cell Reports 15:758-761 (1996). In general, species that are suitable for heterologous pollination belong to the same plant family.

The invention further relates to a plant producing pollen with only one functional sperm cell, and microspores, egg cells, seeds, cells, or tissue from such a plant or progeny thereof.

Finally the invention relates to doubled haploid endosperm, obtainable by means of the method of the invention, as well as to plants regenerated from such double haploid endosperm, progeny of such plants, and to seeds, cells, tissues, microspores and egg cell from such a plant or progeny thereof.

In all embodiments the pollen may contain one functional sperm cell or generative cell which is capable of successfully fertilizing the egg cell.

The present invention will be further illustrated in the following Example which is for illustration purpose only and are not to be construed as limiting this invention in any way.

### EXAMPLE

### Pollination with Mutant Pollen and Endosperm Culture

The CDC2A gene plays a central role in the mitotic cell cycle of plants. A negative mutation in the CDC2A region results in pollen in which mitotic division of the generative cell fails, resulting in pollen with only one sperm cell (Nowack et al., Nature genetics 38: 63 (2006)).

Tomato flowers were emasculated and pollinated with transformed mutant pollen obtained from tomato plants. After pollination, the ovaries expanded and formed fruit-like bodies. The young fruit-like structures were kept on the plants for 2-4 weeks. Plants were grown under climatized conditions (22° C. day, 18° C. night).

Fruits were harvested and the endosperm was separated from the rest of the embryo cells. The endosperms cells were then incubated on a medium commonly used for endosperm regeneration (see T. D. Thomas & R. Chaturvedi, Plant Cell Tissue and Organ Culture 93: 1 (2008) and references therein). Leaf material of successfully generated plants was used to determine the ploidy of the plant by way of flow cytometry (K. E. Arumuganathan & E. D. Earle Plant Molecular Biology Reporter 9: 229). The majority of the plantlets regenerated from endosperm had a nuclear DNA content similar to that of a diploid tomato plant, inferring that these plants are in fact double haploids and that fertilization of the central cell had not taken place.

The invention is further described by the following numbered paragraphs:

1. Method for producing double haploid plants, comprising the steps of:

a) allowing pollen with one functional sperm cell to fertilize an embryo sac cell which is not the central cell;

b) allowing the central cell to proliferate into endosperm; and

c) regenerating a double haploid plant from the endosperm.

2. Method of paragraph 1, wherein the pollen with one functional sperm cell is mutant pollen.

3. Method of paragraph 2, wherein the mutant pollen is obtainable by chemical mutation, transformation with a nucleic acid, or irradiation.

4. Method of paragraph 3 wherein the chemical mutation is effected by treatment of seeds with a chemical agent selected from the group consisting of EMS, EES, BMS, PMS, MES, or MMS.

5. Method of paragraph 3 wherein the irradiation is UV irradiation, X-ray, gamma-ray, or ionizing radiation.

6. Method of paragraph 3 wherein the nucleic acid is either transiently expressed or stably incorporated.

7. Method of paragraph 3, wherein the transformation is performed by means of Agrobacterium tumefaciens or biolistics.

8. Method of paragraph 3, wherein the nucleic acid is or codes for an RNAi which blocks the expression of genes which regulate the formation of a second sperm cell.

9. Method of paragraph 3, wherein the pollen is mutated in a gene involved in inhibiting or arresting the formation of a second sperm cell.

10. Method of paragraph 9, wherein the mutated gene is a negative mutant of the CDC2A or another member of the Cyclin Dependent Kinases protein (CDK) family or a gene of the KRP protein family.

11. Method of paragraph 1, wherein one sperm cell of the pollen grain is destroyed.

12. Method of paragraph 1, wherein a plant producing pollen with one functional sperm cell is obtainable by eco-tilling.

13. Method of any one of the paragraphs 1-12, wherein the pollen with one functional sperm cell are from a donor plant that belongs to another species than the acceptor plant that donates the embryo sac cell or egg cell.

14. Plants producing pollen with only one functional sperm cell according to paragraphs 1 to 13, or progeny, seeds, cells, or tissue from such plants.

15. Double haploid plant or endosperm obtainable by means of a method as paragraphed in any one of the paragraphs 1 to 13.

16. Progeny, seeds, cells, or tissue from endosperm or plants as paragraphed in paragraph 15.

Having thus described in detail preferred embodiments of the present invention, it is to be understood that the invention defined by the above paragraphs is not to be limited to particular details set forth in the above description as many apparent variations thereof are possible without departing from the spirit or scope of the present invention.

What is claimed is:

1. A method for producing a double haploid plant, comprising:
a) allowing pollen with one functional sperm cell to fertilize an embryo sac cell which is not the central cell;
b) allowing the central cell to proliferate into endosperm; and
c) regenerating a double haploid plant from the endosperm.

2. The method of claim 1, wherein the pollen with one functional sperm cell is mutant pollen.

3. The method of claim 2, wherein the mutant pollen is obtainable by chemical mutation, transformation with a nucleic acid, or irradiation.

4. The method of claim 3 wherein the chemical mutation is effected by treatment of seeds with a chemical agent selected from the group consisting of EMS, EES, BMS, PMS, MES, or MMS.

5. The method of claim 3 wherein the irradiation is UV irradiation, X-ray, gamma-ray, or ionizing radiation.

6. The method of claim 3 wherein the nucleic acid is either transiently expressed or stably incorporated.

7. The method of claim 3, wherein the transformation is performed by means of Agrobacterium tumefaciens or biolistics.

8. The method of claim 3, wherein the nucleic acid is or codes for an RNAi which blocks the expression of genes which regulate the formation of a second sperm cell.

9. The method of claim 3, wherein the pollen is mutated in a gene involved in inhibiting or arresting the formation of a second sperm cell.

10. The method of claim 9, wherein the mutated gene is a negative mutant of the CDC2A or another member of the Cyclin Dependent Kinases protein (CDK) family or a gene of the KRP protein family.

11. The method of claim 1, wherein one sperm cell of the pollen grain is destroyed.

12. The method of claim 1, wherein a plant producing pollen with one functional sperm cell is obtainable by eco-tilling.

13. The method of claim 1, wherein the pollen with one functional sperm cell are from a donor plant that belongs to another species than the acceptor plant that donates the embryo sac cell or egg cell.

14. A plant producing pollen with only one functional sperm cell according to the method of claim 1 or progeny, seeds, cells, or tissue from such plants.

15. The progeny, seed, cell or tissue produced from the plant of as claimed in claim 14.

16. A double haploid plant or endosperm obtainable by the method of claim 1.

17. The progeny, seed, cell or tissue from the endosperm or the plant as claimed in claim 16.

18. A method for producing a double haploid plant, comprising regenerating the double haploid plant from endosperm proliferated from the central cell after pollen with one functional sperm cell fertilizes an embryo sac cell which is not the central cell.
